SEQUENCE LISTING (35 sequences)

SEQ ID NO: 1 - DNA, 26 bp, Artificial Sequence
  Synthetic
cctccactcagtgtgattccaccttc

SEQ ID NO: 2 - DNA, 20 bp, Artificial Sequence
  Synthetic
ccactcagtgtgattccacc

SEQ ID NO: 3 - DNA, 25 bp, Artificial Sequence
  Synthetic
gacctccactcagtgtgattccacc

SEQ ID NO: 4 - DNA, 16 bp, Artificial Sequence
  Synthetic
ctcagtgtgattccac

SEQ ID NO: 5 - DNA, 17 bp, Artificial Sequence
  Synthetic
actcagtgtgattccac

SEQ ID NO: 6 - DNA, 20 bp, Artificial Sequence
  Synthetic
tccactcagtgtgattccac

SEQ ID NO: 7 - DNA, 30 bp, Artificial Sequence
  Synthetic
ctcgttgacctccactcagtgtgattccac

SEQ ID NO: 8 - DNA, 20 bp, Artificial Sequence
  Synthetic
ctgaagctgaccctcaggcc

SEQ ID NO: 9 - DNA, 18 bp, Artificial Sequence
  Synthetic
cggggagtgtggtggcag

SEQ ID NO: 10 - DNA, 20 bp, Artificial Sequence
  Synthetic
tgcaggagaccgtactcccc

SEQ ID NO: 11 - DNA, 26 bp, Artificial Sequence
  Synthetic
  misc_feature (2)..(2): The nucleotide in this position is 2′-F-uracil.
  misc_feature (7)..(7): The nucleotide in this position is 2′-F-cytosine.
  misc_feature (12)..(12): The nucleotide in this position is 2′-F-guanidine.
  misc_feature (17)..(17): The nucleotide in this position is 2′-F-guanidine.
  misc_feature (22)..(22): The nucleotide in this position is 2′-F-cytosine.
  misc_feature (26)..(26): The nucleotide in this position is 2′-F-cytosine.
unccacntcagntgtgnattcncacn

SEQ ID NO: 12 - DNA, 30 bp, Artificial Sequence
  Synthetic
gcgccgggcctttctttatgtttttggcgt

SEQ ID NO: 13 - DNA, 20 bp, Artificial Sequence
  Synthetic
gagaaagacaatatagttct

SEQ ID NO: 14 - DNA, 127 bp, Artificial Sequence
  Synthetic
gcaaatgcttgctagaccaataattagttattcaccttgctaaagaaattcttgctcgttgacctccactcagtgtgattccaccttctcaaagaactatattgtctttctctgcaaacttggagat

SEQ ID NO: 15 - DNA, 21 bp, Artificial Sequence
  Synthetic
atggaagcccagtgaagatac

SEQ ID NO: 16 - DNA, 20 bp, Artificial Sequence
  Synthetic
ctagccataaaaccccagga

SEQ ID NO: 17 - DNA, 26 bp, Artificial Sequence
  Synthetic
  misc_feature (1)..(8): n is a, c, g, or t
  misc_feature (12)..(26): n is a, c, g, or t
nnnnnnnntgannnnnnnnnnnnnnn

SEQ ID NO: 18 - DNA, 65 bp, Artificial Sequence
  Synthetic
atgagaaacaatgacaagacgccaaaaacataaagaaaggcccggcgccattctatcctctagag

SEQ ID NO: 19 - DNA, 30 bp, Artificial Sequence
  Synthetic
tgcggtttttgtatttctttccgggccgcg

SEQ ID NO: 20 - DNA, 62 bp, Artificial Sequence
  Synthetic
aagacaatatagttctttgagaaggtggaatcacactgagtggaggtcaacgagcaagaatt

SEQ ID NO: 21 - DNA, 30 bp, Artificial Sequence
  Synthetic
caccttagtgtgactcacctccagttgctc

SEQ ID NO: 22 - DNA, 20 bp, Artificial Sequence
  Synthetic
tccactcagtgtgattccac

SEQ ID NO: 23 - DNA, 29 bp, Artificial Sequence
  Synthetic
gcaatgaaaataaatttcctttattagcc

SEQ ID NO: 24 - DNA, 28 bp, Artificial Sequence
  Synthetic
gacaatatagttcttggagaaggtggaa

SEQ ID NO: 25 - DNA, 28 bp, Artificial Sequence
  Synthetic
gacaatatagttctttgagaaggtggaa

SEQ ID NO: 26 - DNA, 29 bp, Artificial Sequence
  Synthetic
gacaatatagttctttgagaaggtggaat

SEQ ID NO: 27 - DNA, 43 bp, Artificial Sequence
  Synthetic
agatgcgatctgtgagccgagtctttaagttcattgacatgcc

SEQ ID NO: 28 - DNA, 43 bp, Artificial Sequence
  Synthetic
agatgcgatctgtgagctgagtctttaagttcattgacatgcc

SEQ ID NO: 29 - DNA, 43 bp, Artificial Sequence; identical to SEQ ID NO: 28
  Synthetic

SEQ ID NO: 30 - DNA, 180 bp, Artificial Sequence
  Synthetic
ttatcagcttttttgagactactgaacactgaaggagaaatccagatcgatggtgtgtcttgggattcaataactttgcaacagtggaggaaagcctttggagtgataccacaggtgagcaaaaggacttagccagaaaaaaggcaactaaattatattttttactgctatttgatactt

SEQ ID NO: 31 - DNA, 180 bp, Artificial Sequence
  Synthetic
ttatcagcttttttgagactactgaacactgaaggagaaatccagatcgatggtgtgtcttgggattcaataactttgcaacagtgaaggaaagcctttggagtgataccacaggtgagcaaaaggacttagccagaaaaaaggcaactaaattatattttttactgctatttgatactt

SEQ ID NO: 32 - DNA, 28 bp, Artificial Sequence
  Synthetic
ctatcgcgatttatctaggcataggctt

SEQ ID NO: 33 - DNA, 28 bp, Artificial Sequence
  Synthetic
ctatcgcgatttaactaggcataggctt

SEQ ID NO: 34 - DNA, 28 bp, Artificial Sequence
  Synthetic
ctatcgcgatttaactaggcataggctt

SEQ ID NO: 35 - DNA, 21 bp, Artificial Sequence
  Synthetic
  misc_feature (21)..(21): The nucleotide in this position is 2′-F-uracil/2′-F-cytosine/2′-F-guanidine.
  misc_feature (21)..(21): The nucleotide in this position is 2′-F-cytosine.
tccactcagtgtgattccacn

(19) United States
(12) Patent Application Publication
(10) Pub. No.: US 2024/0209376 A1
(43) Pub. Date: Jun. 27, 2024

(54) Specific Oligonucleotide-Programmed Readthrough of Nonsense Codons

(71) Applicant: UNIVERSITY OF MASSACHUSETTS, Boston, MA (US)

(72) Inventors: Denis Susorov, Worcester, MA (US); Andrei Korostelev, Shrewsbury, MA (US); Seraj Zahra, Worcester, MA (US)

(21) Appl. No.: 18/553,357

(22) PCT Filed: Apr. 6, 2022
(86) PCT No.: PCT/US2022/023640

§ 371 Date: Sep. 29, 2023

Related U.S. Application Data
(60) Provisional application No. 63/171,893, filed on Apr. 7, 2021.

(51) Int. Cl. C12N 15/113

(52) U.S. Cl. CPC C12N 15/1138; C12N 2310/11; C12N 2310/315; C12N 2310/321; C12N 2310/322

(57) ABSTRACT

This invention is related to the field of genetic engineering. In particular, it is related to compositions and methods to treat genetically-based diseases and disorders. For example, nucleic acid oligomers are contemplated that promote translation readthrough of premature stop codons that produce non-functional proteins. DNA and modified nucleic acid oligos that bind at the +4 through +8 (+4, +5, +6, +7 and +8) nucleotide position downstream of a premature stop codon successfully promoted readthrough of a premature stop codon in a cystic fibrosis gene.

### STATEMENT OF GOVERNMENTAL SUPPORT

This invention was made with government support under GM127094 awarded by The National Institutes of Health. The government has certain rights in the invention.

### FIELD OF THE INVENTION

This invention is related to the field of genetic engineering. In particular, it is related to compositions and methods to treat genetically-based diseases and disorders that are caused by the translation of non-functional proteins from an mRNA with a nonsense stop codon. For example, DNA and modified nucleic acid (NA) oligomers are contemplated that promote translation readthrough of premature mRNA stop (nonsense) codons. Nonsense codon readthrough results in a full-length protein and restores protein function. For example, DNA oligomers that bind starting at a +4 through +8 nucleotide position (i.e., +4, +5, +6, +7, +8 positions) downstream from the first nucleotide of a premature mRNA stop codon (+1 position, see FIG. 1) successfully promoted readthrough.

### BACKGROUND

Mutations resulting in premature nonsense (i.e., stop) codons lead to more than 10% of human genetic diseases. Stop codons normally result in the physical release of proteins from the ribosome. Thus, a premature stop codon results in the translation of a short (e.g. truncated), often non-functional protein, resulting in disease.

Most tested drugs (e.g. aminoglycosides, such as G418) can provide global readthrough of nonsense or stop codons but induce miscoding for many cellular mRNAs, thereby resulting in a clinically unacceptable level of toxicity.

What is needed in the art are compositions and methods to increase readthrough of a premature stop codon in a specific mRNA molecule.

### SUMMARY OF THE INVENTION

This invention is related to the field of genetic engineering. In particular, it is related to compositions and methods to treat genetically-based diseases and disorders that are caused by the translation of non-functional proteins from an mRNA with a nonsense stop codon. For example, DNA and modified nucleic acid (NA) oligomers are contemplated that promote translation readthrough of premature mRNA stop (nonsense) codons. Nonsense codon readthrough results in full-length protein and restores protein function. For example, DNA oligomers that bind starting at a +4 through +8 nucleotide position (i.e., +4, +5, +6, +7, +8 positions) downstream from the first nucleotide of a premature mRNA stop codon (+1, see FIG. 1) successfully promoted readthrough. In comparison, nucleotides placed further downstream of the first nucleotide of the first nonsense stop codon (+9 position, etc.) did not promote readthrough.

In one embodiment, the present invention contemplates a deoxyribonucleic (DNA) antisense oligomer that is complementary to a ribonucleic acid (RNA) sequence starting between a +4-+8 nucleotide position downstream of the first nucleotide in a premature stop codon within a messenger RNA (mRNA) molecule. In one embodiment, the premature stop codon is UGAC (RNA). In one embodiment, the premature stop codon is UGAG. In one embodiment, the premature stop codon is UGAA. In one embodiment, the premature stop codon is UGAU. In one embodiment, the DNA oligomer has a melting temperature (Tₘ) including, but not limited to, 32° C., 34° C., 40° C., 47° C., 53° C., 57° C. or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)). In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)).
In one embodiment, the DNA oligomer is CTGAAGCTGACCCTCAGGCC (Mecp2 +8(57) 255X). In one embodiment, the DNA oligomer is CGGGGAGTGTGGTGGCAG (Mecp2 +8(57) 270X). In one embodiment, the DNA oligomer is TGCAGGAGACCGTACTCCCC (Mecp2 +8(57) 294X). In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a composition comprising: i) a deoxyribonucleic (DNA) or modified nucleic acid antisense oligomer; and ii) a messenger ribonucleic acid (mRNA) molecule encoding an RNA sequence and a premature stop codon, wherein the antisense oligomer is complementary to the RNA sequence starting between a +4-+8 nucleotide position downstream of the first nucleotide of the premature stop codon. In one embodiment, the antisense oligomer is hybridized to the RNA sequence. In one embodiment, the mRNA molecule encodes a cystic fibrosis transmembrane conductance regulator protein (CFTR). In one embodiment, the mRNA molecule encodes a methylcytosine-binding protein 2 (Mecp2). In one embodiment, the composition further comprises an aminoglycoside. In one embodiment, the composition further comprises an aminoglycoside concentration that is 5-fold lower than a conventional aminoglycoside concentration that promotes translation readthrough. In one embodiment, the aminoglycoside is G418. In one embodiment, the aminoglycoside includes, but is not limited to, gentamicin, amikacin, tobramycin, kanamycin, streptomycin or neomycin. In one embodiment, the premature stop codon is UGAC. In one embodiment, the premature stop codon is UGAG. In one embodiment, the premature stop codon is UGAA. In one embodiment, the premature stop codon is UGAT. In one embodiment, the DNA oligomer has a melting temperature (Tₘ) including, but not limited to, 32° C., 34° C., 40° C., 47° C., 53° C., or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)). In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)). In one embodiment, the DNA oligomer is CTGAAGCTGACCCTCAGGCC (Mecp2 +8(57) 255X). In one embodiment, the DNA oligomer is CGGGGAGTGTGGTGGCAG (Mecp2 +8(57) 270X) In one embodiment, the DNA oligomer is TGCAGGAGACCGTACTCCCC (Mecp2 +8(57) 294X) In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a method, comprising: a) providing; i) a patient comprising a messenger ribonucleic acid (mRNA) molecule with a premature stop codon and exhibiting at least one symptom of medical disorder; and ii) a pharmaceutically acceptable composition comprising a deoxyribonucleic (DNA) or modified nucleic acid antisense oligomer that is complementary to a mRNA sequence starting between a +4-+8 nucleotide position downstream of the first nucleotide of the premature stop codon; and b) administering the pharmaceutically acceptable composition to the patient such that said at least one symptom of said medical disorder is reduced. In one embodiment, the medical disorder is caused by the premature stop codon. In one embodiment, the medical disorder is cystic fibrosis or Rett syndrome. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA molecule encodes a cystic fibrosis transmembrane conductance regulator protein. In one embodiment, the mRNA molecule encodes a methylcytosine-binding protein 2. In one embodiment, the premature stop codon is UGAC. In one embodiment, the premature stop codon is UGAG. In one embodiment, the premature stop codon is UGAA. In one embodiment, the premature stop codon is UGAT. In one embodiment, the DNA oligomer has a melting temperature (Tₘ) including, but not limited to, 32° C., 34° C., 40° C., 47° C., 53° C., or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)). In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)). In one embodiment, the DNA oligomer is CTGAAGCTGACCCTCAGGCC (Mecp2 +8(57) 255X). In one embodiment, the DNA oligomer is CGGGGAGTGTGGTGGCAG (Mecp2 +8(57) 270X). In one embodiment, the DNA oligomer is TGCAGGAGACCGTACTCCCC (Mecp2+$(57) 294X). In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a method, comprising: a) providing; i) a patient comprising a messenger ribonucleic acid (mRNA) molecule with a premature stop codon and exhibiting at least one symptom of medical disorder; and ii) a pharmaceutically acceptable composition comprising an aminoglycoside and a deoxyribonucleic (DNA) or modified nucleic acid antisense oligomer that is complementary to an mRNA sequence starting between a +4-+8 nucleotide position downstream of the first nucleotide of said premature stop codon; and b) administering the pharmaceutically acceptable composition to the patient such that said at least one symptom of said medical disorder is reduced. In one embodiment, the administering has a synergistic reduction of said at least one symptom as compared to said aminoglycoside alone. In one embodiment, the medical disorder is caused by the premature stop codon. In one embodiment, the medical disorder is cystic fibrosis or Rett syndrome. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA molecule encodes a cystic fibrosis transmembrane conductance regulator protein. In one embodiment, the mRNA molecule encodes a methylcytosine-binding protein 2. In one embodiment, the premature stop codon is UGAC. In one embodiment, the premature stop codon is UGAG. In one embodiment, the premature stop codon is UGAA. In one embodiment, the premature stop codon is UGAT. In one embodiment, the DNA oligomer has a melting temperature (Tₘ) including, but not limited to, 32° C., 34° C., 40° C. 47° C., 53° C., 66° C. or 70° C. In one embodiment, the DNA oligomer has a melting temperature (Tₘ) including, but not limited to, 32° C., 34° C., 40° C., 47° C., 53° C., or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)).

In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)). In one embodiment, the DNA oligomer is CTGAAGCTGACCCTCAGGCC (Mecp2 +8(57) 255X). In one embodiment, the DNA oligomer is CGGGGAGTGTGGTGGCAG (Mecp2 +8(57) 270X). In one embodiment, the DNA oligomer is TGCAGGAGACCGTACTCCCC (Mecp2 +8(57) 294X). In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a method, comprising: a) providing; i) a patient exhibiting at least one symptom of cystic fibrosis; and ii) a pharmaceutically acceptable composition comprising a deoxyribonucleic (DNA) or modified nucleic acid antisense oligomer that is complementary to a messenger ribonucleic acid (mRNA) sequence starting between a +4-+8 nucleotide position downstream of the first nucleotide of a premature stop codon within an mRNA molecule; and b) administering the pharmaceutically acceptable composition to the patient such that said at least one symptom of cystic fibrosis is reduced. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a cystic fibrosis transmembrane conductance regulator protein. In one embodiment, the premature stop codon is UGAC. In one embodiment, the premature stop codon is UGAG. In one embodiment, the premature stop codon is UGAA. In one embodiment, the premature stop codon is UGAT. In one embodiment, the DNA oligomer has a melting temperature (Tₘ) including, but not limited to, 32° C., 34° C., 40° C., 47° C., 53° C., or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)). In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)). In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a method, comprising: a) providing; i) a patient exhibiting at least one symptom of Rett syndrome; and ii) a pharmaceutically acceptable composition comprising a deoxyribonucleic (DNA) or modified nucleic acid antisense oligomer that is complementary to a messenger ribonucleic acid (mRNA) sequence starting between a +4-+8 nucleotide position downstream of the first nucleotide of a premature stop codon within an mRNA molecule; and b) administering the pharmaceutically acceptable composition to the patient such that said at least one symptom of Rett Syndrome is reduced. In one embodiment, the pharmaceutically acceptable composition further comprises an aminoglycoside. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a methylcytosine-binding protein 2. In one embodiment, the premature stop codon is UGAA. In one embodiment, the premature stop codon is UGAT. In one embodiment, the DNA oligomer is CTGAAGCTGACCCTCAGGCC (Mecp2 +8(57) 255X). In one embodiment, the DNA oligomer is CGGGGAGTGTGGTOGCAG (Mecp2 +8(57) 270X) In one embodiment, the DNA oligomer is TGCAGGAGACCGTACTCCCC (Mecp2 +8(57) 294X)

In one embodiment, the present invention contemplates a method, comprising: a) providing; i) a patient exhibiting at least one symptom of cystic fibrosis; and ii) a pharmaceutically acceptable composition comprising an aminoglycoside and a pharmaceutically acceptable composition comprising a deoxyribonucleic (DNA) or modified nucleic acid antisense oligomer that is complementary to a messenger ribonucleic acid (mRNA) sequence starting between a +4-+8 nucleotide position downstream of the first nucleotide of a premature stop codon within an mRNA molecule; and b) administering the pharmaceutically acceptable composition to the patient such that said at least one symptom of cystic fibrosis is reduced. In one embodiment, the administering has a synergistic reduction of said at least one symptom as compared to said aminoglycoside alone. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a cystic fibrosis transmembrane conductance regulator protein. In one embodiment, the premature stop codon is UGAC. In one embodiment, the premature stop codon is UGAG. In one embodiment, the DNA oligomer has a melting temperature (Tₘ) including, but not limited to, 32° C., 34° C., 40° C., 47° C., 53° C., or 66° C. In one embodiment, the DNA oligomer is CCTCCACTCAGTGTGATTCCACCTTC (CFTR +4(60)). In one embodiment, the DNA oligomer is CCACTCAGTGTGATTCCACC (CFTR +7(49)). In one embodiment, the DNA oligomer is GACCTCCACTCAGTGTGATTCCACC (CFTR +7 (60)). In one embodiment, the DNA oligomer is CTCAGTGTGATTCCAC (CFTR +8(32)). In one embodiment, the DNA oligomer is ACTCAGTGTGATTCCAC (CFTR +8(34)). In one embodiment, the DNA oligomer is TCCACTCAGTGTGATTCCAC (CFTR +8(47)). In one embodiment, the DNA oligomer is CTCGTTGACCTCCACTCAGTGTGATTCCAC (CFTR +8(66)). In one embodiment, the antisense oligomer comprises at least one nucleotide with a modification. In one embodiment, these modifications include, but are not limited to, a 2′-fluoride (F) modification, a 2′-O-methyl (Ome) modification and a phosphothioate (PS) linkage modification. In one embodiment, the antisense oligomer is a CFTR +8(47) oligo comprising a fluoride modified CFTR +8(47) oligo. In one embodiment, the fluoride modified CFTR +8(47) oligomer is TCCACTCAGTGTGATTCCAC^{F}. In one embodiment, the fluoride modified CFTR +8(47) oligomer is U^{F}CCAC^{F}TCAG^{F}TGTG^{F}ATTC^{F}CAC^{F}.

In one embodiment, the present invention contemplates a method, comprising: a) providing; i) a patient exhibiting at least one symptom of Rett syndrome; and ii) a pharmaceutically acceptable composition comprising an aminoglycoside and a deoxyribonucleic (DNA) antisense oligomer that is complementary to a messenger ribonucleic acid (mRNA) sequence starting between a +4-+8 nucleotide position downstream of the first nucleotide of a premature stop codon within an mRNA molecule; and b) administering the pharmaceutically acceptable composition to the patient such that said at least one symptom of Rett Syndrome is reduced. In one embodiment, the administering has a synergistic reduction of said at least one symptom as compared to said aminoglycoside alone. In one embodiment, the administering does not result in aminoglycoside side effects. In one embodiment, the aminoglycoside is G418. In one embodiment, the aminoglycoside includes, but is not limited to, gentamicin, amikacin, tobramycin, kanamycin, streptomycin or neomycin. In one embodiment, the mRNA sequence encodes a methylcytosine-binding protein 2. In one embodiment, the premature stop codon is UGAC. In one embodiment, the premature stop codon is UGAG. In one embodiment, the DNA oligomer has a melting temperature (Tₘ) including, but not limited to, 57° C. In one embodiment, the premature stop codon is UGAA. In one embodiment, the premature stop codon is UGAT. In one embodiment, the DNA oligomer is CTGAAGCTGACCCTCAGGCC (Mecp2 +8(57) 25SX). In one embodiment, the DNA oligomer is CGGGGAGTGTGGTGGCAG (Mecp2 +8(57) 270X). In one embodiment, the DNA oligomer is TGCAGGAGACCGTACTCCCC (Mecp2 +8(57) 294X).

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The generic term “+#(##)” as used herein, refers to an antisense nucleotide binding nomenclature format. For example, the antisense oligonucleotide designation of “+8(70)” means that the “+8” refers to the registered binding nucleotide which is eight (8) nucleotides downstream of the first nucleotide of a nonsense/premature stop codon and the “(70)” is the melting temperature for an analogous DNA-DNA duplex having the same sequence of the antisense oligo. The other antisense oligo designations presented herein follow the same format and interpretation.

The term “aminoglycoside” as used herein, refers to any organic molecule that contains amino sugar substructures. Clinically, an aminoglycoside is a medicinal and bacteriologic category of traditional gram-negative antibacterial medications that inhibit protein synthesis and contain as a portion of the molecule an amino-modified glycoside. For example, an aminoglycoside includes, but is not limited to, G418, gentamicin, amikacin, tobramycin, kanamycin, streptomycin and neomycin. It is generally known that the administration of conventional concentrations (i.e., doses) of an aminoglycoside results in side effects in a large percentage of patients. Such side effects include those systems related to, but are not limited to, auditory, renal and vestibular.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” as used herein, refers to an art-accepted causal relationship between a genetic mutation and a medical condition or disease. For example, it is art-accepted that a patient having an CTFR gene comprising a mutation that generates a premature stop codon has, or is a risk for, cystic fibrosis.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA or their modified analogs of genomic or synthetic origin which may be single- or double-stranded, and represent the sense or antisense strand.

The term “modified nucleic acid”, as used herein, refers to any nucleic acid molecule having modified backbone, sugar, nucleobase, or novel base or base pair.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

The term “nucleic acid sequence” and “nucleotide sequence” as used herein, refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to an amino acid sequence refers to fragments of that amino acid sequence. The fragment may range in size from 2 amino acid residues to the entire amino acid sequence minus one amino acid residue.

As used herein, the term “antisense” is used in reference to nucleic acid sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “functionally equivalent codon”, as used herein, refers to different codons that encode the same amino acid. This phenomenon is often referred to as “degeneracy” of the genetic code. For example, six different codons encode the amino acid arginine.

A “variant” of a protein is defined as an amino acid sequence which differs by one or more amino acids from a polypeptide sequence or any homolog of the polypeptide sequence. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions (i.e., additions), or both. Guidance in determining which and how many amino acid residues may be substituted, inserted or deleted without abolishing biological or immunological activity may be found using computer programs including, but not limited to, DNAStar® software.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues.

A “substitution” results from the replacement of one or more nucleotides or amino acids by different nucleotides or amino acids, respectively.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization”, “hybridized” or “hybridizing” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Co tor Ro t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tₘ” is used in reference to the “melting temperature.” The melting temperature is the temperature at which a population of double-stranded nucleic acid molecules becomes half dissociated into single strands. As indicated by standard references, a simple estimate of the Tₘ value may be calculated by the equation: Tₘ=81.5+0.41 (% G+C), when a nucleic acid is in aqueous solution at 1M NaCl. Anderson et al., “Quantitative Filter Hybridization” In: Nucleic Acid Hybridization (1985). More sophisticated computations take structural, as well as sequence characteristics, into account for the calculation of Tₘ.

The term “DNA oligomers” are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “nucleic acid molecule encoding”, “RNA sequence encoding”, “DNA sequence encoding,” and “DNA encoding” refer to the order or sequence of (deoxy)ribonucleotides along a strand of (deoxy)ribonucleic acid. The order of these (deoxy)ribonucleotides determines the order of amino acids along the polypeptide (protein) chain. The DNA and RNA sequences thus code for an amino acid sequence.

As used herein, the term “coding region” or “open reading frame (ORF)” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents a clarification of different nucleotide register nomenclatures between that used to describe the present invention and that disclosed in Kar et al., “Induction of Translational Readthrough across the Thalassemia-Causing Premature Stop Codon in ß-Globin-Encoding mRNA” Biochemistry 59(1):80-84 (2020; online Oct. 2, 2019).

FIG. 2 presents an exemplary illustration of translation of a hypothetical protein from an mRNA molecule (black) with either a premature (nonsense) stop codon or a natural (wild type) stop codon.

FIG. 2A: A truncated protein (blue, left) is produced at a premature stop codon (red, left). A full length protein (blue, right) is produced at a natural stop codon (blue left).

FIG. 2B: An illustration of termination factors (green) which recognizes and binds to either a premature stop codon (red, left) or a natural stop codon (red, right). The termination factor sterically “pulls” the stop codon into ribosome (gray), thereby releasing the translated protein (blue).

FIG. 2C: An illustration of a site-specific antisense DNA or modified oligo (light green) positioned downstream of a premature stop codon (red, left) that interferes with a steric “pull” or other interaction with the termination factor (green) on the ribosome (gray), thereby allowing a readthrough of the complete mRNA and translation of a full length protein.

FIG. 3 illustrates a rabbit reticulocyte luciferase mRNA translation assay.

FIG. 3A: Step-wise illustration showing the translation of a luciferase mRNA molecule (black) with a stop codon (red) into full length luciferase protein (blue) that emits light (arrows).

FIG. 3B: Exemplary data of light intensity (e.g., relative luminescence units (RLUs)) during luciferase mRNA translation. Left panel: The light intensity pattern as a function of time (e.g., seconds). Middle panel: The rate of luciferase translation shown by light intensity fluctuation over time. Right panel: Depicts the maximal achievable rate of the luciferase translation shown by light intensity fluctuation over time.

FIG. 4 illustrates one embodiment of a CAN1 arginine permease gene/luciferase expression construct (Can1-1uc). Upper panel: A schematic of the construct showing the relative position of a premature stop codon and a Can1 open reading frame and a luciferase gene with a natural terminal stop codon. Lower panel: A deoxyribonucleic acid sequence of the Can1-1uc construct with a TGA premature stop codon and a deoxyribonucleic acid oligo hybridized to the Can1-1uc construct at the +8 nucleotide position downstream of the premature stop codon.

FIG. 5 presents exemplary data showing readthrough of a CAN1-1uc expression construct premature stop codon with a DNA oligo hybridized at the +8 nucleotide position downstream of the premature stop codon.

FIG. 6 presents one embodiment of a cystic fibrosis gene/nanoluciferase expression construct (511-565 CFTR). Upper panel: A schematic of the construct showing the relative position of a premature stop codon (UGAG or UGAC) within a cystic fibrosis open reading frame (CFTR) and a luciferase gene with a natural terminal stop codon. Lower panel: A deoxyribonucleic acid sequence of the CFTR construct with a TGA premature stop codon and a deoxyribonucleic acid oligo hybridized to the CFTR construct at the +8 nucleotide position downstream of the premature stop codon.

FIG. 7 presents exemplary data showing a readthrough analysis of a CFTR premature stop codon (UGAC) construct expression in the presence of a DNA oligo targeted to various nucleotide positions downstream of the premature stop codon as well as having different melting temperatures.

FIG. 8 presents exemplary data showing the effect of the aminoglycoside G418 on 511-565 CFTR construct expression. Increasing concentrations of G418 decreases the expression of wild type construct without premature stop codon (GGAG) while increasing expression of premature stop codon constructs (UGAG, UGAC).

FIG. 9 presents exemplary data showing the synergistic effect of a +8(66) DNA oligo and the aminoglycoside G418 on CFTR DNA construct expression readthrough of premature stop codons. Also shown is a +8 oligo in combination with G418 that decreases the effective concentration for an aminoglycoside.

FIG. 10 presents exemplary data showing a synergistic effect of a +8(47) DNA oligo and the aminoglycoside G418 on CFTR DNA construct expression readthrough of premature stop codons.

FIG. 11 presents exemplary data correlating nanoluciferase activity with expressed protein level to validate readthrough promotion by the oligo constructs as contemplated herein.

FIG. 12 provides a schematic of a luciferase-based assay (TermiLuc, Susorov, 2020) to identify the loss of a translation termination step to promote readthrough.

FIG. 12A: A schematic of the Termi-Luc assay.

FIG. 12B: One example of a eukaryotic termination complex.

FIG. 13 presents exemplary data showing that readthrough promoting oligos +7 and +8 inhibit termination in a sequence specific-manner in the Termi-Luc assay.

FIG. 14 presents an illustrative structure of a modified oligo with a 2′-fluoride substitution.

FIG. 15 presents exemplary data showing readthrough promotion with fluoride-modified oligos using a rabbit reticulocyte lysate (RRL) assay.

FIG. 16 presents exemplary data showing promotion of CFTR nonsense codon readthrough by a combination of G418 and a modified antisense oligonucleotide in a dose-dependent manner. O=modified oligo; G=G418 in the culture of cell expressing CFTR with premature stop codon G542X, fused with HRP to measure chemiluminescence resulting from full-length protein expression.

FIG. 17 presents one embodiment of a full length Mecp2 gene/nanoluciferase expression construct.

FIG. 17A: A schematic of the construct showing the relative position of a premature stop codon within a full length Mecp2 open reading frame and a luciferase gene with a natural terminal stop codon.

FIG. 17B: Exemplary data showing the effect of a +8 DNA oligo on readthrough efficiency for four (4) premature stop codons responsible for Rett syndrome.

FIGS. 18A-D present exemplary data showing next-generation bulk sequencing results of PCR amplicons using TOPO cloning (e.g., allelic exclusion) method.

FIGS. 19A-F present exemplary data showing an analysis of gene expression using both quantitative polymerase chain reaction and Western Blot.

FIGS. 20A-C present exemplary data of a TECC/24 conductance assay performed at one (1) week post-filter seeding.

FIGS. 21A-B present exemplary data of transepithelial resistance of the CFF-16HBEge cell lines.

### DETAILED DESCRIPTION OF THE INVENTION

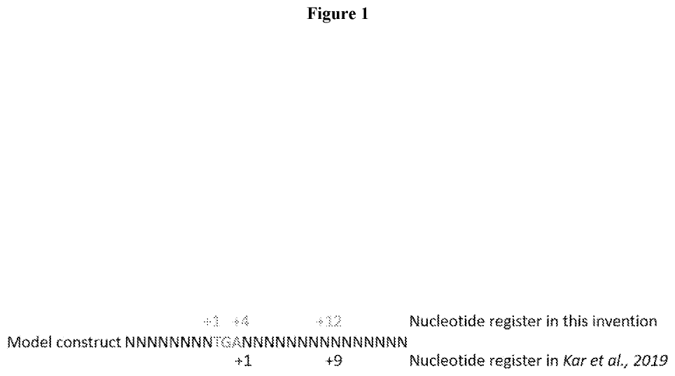

This invention is related to the field of genetic engineering. In particular, it is related to compositions and methods to treat genetically-based diseases and disorders that are caused by the translation of non-functional proteins from an mRNA with a nonsense stop codon. For example, DNA and modified nucleic acid (NA) oligomers are contemplated that promote translation readthrough of premature mRNA stop (nonsense) codons. Nonsense codon readthrough results in a full-length protein and restores protein function. For example, DNA oligomers that bind starting at a +4 through +8 nucleotide position (i.e., +4, +5, +6, +7, +8 positions) downstream from the first nucleotide of a premature mRNA stop codon (+1, see FIG. 1) successfully promoted readthrough.

In one embodiment, the present invention contemplates a method to treat a genetic disorder caused by a premature nonsense or stop codons. In one embodiment, the genetic disorder includes, but is not limited to, cystic fibrosis (CF) or Rett syndrome. In one embodiment, the nonsense or stop codon is an X-stop codon. In one embodiment the X-stop codon is G542X. In one embodiment the X-stop is R255X (Rett). In one embodiment the X-stop is R270X (Rett). In one embodiment the X-stop is R294X (Rett).

### II. Conventional Stop Codon Readthrough Methods

There have been numerous efforts to identify therapeutics that would readthrough a premature stop codon and restore full-length protein translation. Small molecules, such as aminoglycoside antibiotics, result in systematic miscoding of mRNAs and are of limited therapeutic value. Although they also result in stop-codon readthrough with a mutant mRNA, broad miscoding of cellular mRNAs makes such molecules toxic and generally poor therapeutic agents. Dabrowski et al. “Advances in therapeutic use of a drug-stimulated translational readthrough of premature termination codons” Mol Med 24 (2018); and Keeling et al., “Therapeutics Based on Stop Codon Readthrough”. Annu Rev Genomics Hum Genet. (2014).

The main reported adverse effects of aminoglycosides are ototoxicity, nephrotoxicity, and neuromuscular blockade. Avent et al., “Current use of aminoglycosides: indications, pharmacokinetics and monitoring for toxicity” Intern Med J. 41(6):441-449 (2011). Conventional dosing of an aminoglycoside is usually about 3-5 mg/kg/day given intravenously/intramuscularly (IV/IM) divided every 8 hours. An extended dosing interval is about every 24 hours or more at 4-7 mg/kg/dose IV once/day.

Aminoglycoside antibiotics are used as a conventional treatment of pulmonary exacerbations of cystic fibrosis (CF) and slow the decline in lung function which ultimately causes the death of most patients. The prognosis of CF has improved, and thus side effects of treatments have become increasingly important. Prayle et al., “Side effects of aminoglycosides on the kidney, ear and balance in cystic fibrosis” Thorax 65(7):654-658 (2010).

Observational studies suggest that the morbidity from side effects of aminoglycosides is disturbingly common, and that aggressive treatment may lead to more side effects. One common side effect of aminoglycosides is renal toxicity. Studies vary in their definition of toxicity, but approximately 5-10% of (non-CF) adult patients receiving an aminoglycoside have a significant increase in serum creatinine. Meyer R D., “Risk factors and comparisons of clinical nephrotoxicity of aminoglycosides” Am J Med 80:119-125 (1986).

Although toxicity is an aminoglycoside class effect, experimental data suggest that gentamicin is more toxic than tobramycin and amikacin. De Broe et al., “Choice of drug and dosage regimen. Two important risk factors for aminoglycoside nephrotoxicity” Am J Med 80:115-118 (1986). A quantitative overview of randomised controlled trials (RCTs) reached broadly the same conclusions. Buring et al., “Randomized trials of aminoglycoside antibiotics: quantitative overview” Rev Infect Dis 10:951-7 (1988).

Manipulation of an aminoglycoside dosing regimen provides a cost-effective and simple method of reducing kidney injury. Given the saturable uptake of aminoglycosides, it has been reported that a single daily dose would be expected to be less nephrotoxic than the same daily dose in three divided doses. For example, a large randomised trial of tobramycin for patients with CF, established that there is equal efficacy with a single daily dosing regimen as with a multiple daily dosing regimen, a finding confirmed in a subsequent meta-analysis. In a paediatric group receiving a single daily dose, the serum creatinine level decreased during the course of treatment compared with a rise in the group receiving three divided doses. In further support of renal safety, in the once daily arm the rise in NAG was 33% less than in the group receiving a three times daily regimen in both adults and children. Smyth et al., “Once versus three-times daily regimens of tobramycin treatment for pulmonary exacerbations of cystic fibrosis—the TOPIC study: a randomised controlled trial” Lancet 365:573-578 (2008); and Smyth et al., “Once-daily versus multiple-daily dosing with intravenous aminoglycosides for cystic fibrosis” Cochrane Database Syst Rev 2006; (3):CD002009. The pharmacokinetics of aminoglycosides are complicated by a circadian rhythm in elimination. In the once daily group, most of whom received their antibiotics in the evening, there was a lower elimination rate of tobramycin than in the three times daily group. Touw et al., “Population pharmacokinetics of tobramycin administered thrice daily and once daily in children and adults with cystic fibrosis” J Cyst Fibros 6:327-333 (2007) There may be a diurnal variation in renal clearance of the drug, with decreased clearance occurring at night. This would lead to increased exposure of the kidneys to aminoglycoside during the course of the illness if the drug is administered at night compared with the morning.

A selection of the numerous studies report auditory toxicity as measured by Pure Tone Audigrams (PTAs) in CF patients. For example, a study of seventy (70) patients recruited from a CF clinic and, using a definition of ≥2 thresholds of >20 dB or one of >25 dB, they found an overall prevalence of hearing impairment of 17% when given aminoglycosides. Mulheran et al., “Occurrence and risk of cochleotoxicity in cystic fibrosis patients receiving repeated high-dose aminoglycoside therapy” Antimicrob Agents Chemother 45:2502-2509 (2001).

It has been reported that DNA oligonucleotides complementary to a beta-globin mRNA at +1 or +9 nucleotides downstream of an artificially introduced premature stop codon “UAG” can induce translational readthrough in cells. Kar et al., “Induction of Translational Readthrough across the Thalassemia-Causing Premature Stop Codon in β-Globin-Encoding mRNA” Biochemistry 59(1):80-84 (2020; online Oct. 2, 2019). However, these DNA oligonucleotides have not been shown to be an effective therapeutic for any known genetic disorder.

It should be noted that the DNA oligo starting position nomenclature system of Kar et al. differs from that used in the present invention, and the DNA oligo positions used by Kar et al do not apply to CFTR and Mecp2. In particular, Kar's +1 and +9 positions correspond to the +4 and +12 positions (respectively) as presented herein (see FIG. 1). Consequently, DNA oligos that are complementary to an mRNA sequence starting at the +5-+8 nucleotide position downstream of the first nucleotide of a premature stop codon have not been previously reported. In particular, oligonucleotides complementary for the CFTR and Mecp2 genes at the +4-+8 nucleotide position downstream of the first nucleotide of a premature stop codon have not been previously reported. For example, nucleotides placed further downstream of the first nucleotide of the nonsense codon (e.g., positions +9, +12) did not promote readthrough for CFTR.

### II. Nonsense Codon-Associated Protein Release Factors

In one embodiment, the present invention contemplates compositions and methods to induce efficient mRNA-specific readthrough of premature stop (nonsense) codons resulting in minimal off-target side effects. Although it is not necessary to understand the mechanism of an invention, it is believed that such an approach relies on structural differences between the cellular recognition of stop codons and sense codons.

For example, cellular recognition of stop codons is believed to be mediated by protein release factors (i.e., eRF1 in eukaryotes). Recognition occurs in an A-site codon where an eRF1 protein interacts with an mRNA nucleotide sequence at a stop codon and the following nucleotide (for example, UGAC or UAAA). By contrast, triplet sense codons are recognized by tRNA, where the mRNA sequence downstream of the A-site codon is then threaded through a ribosomal mRNA tunnel and exits into solution. Thus, an eRF1 recognition protein requires that an mRNA be “pulled” into the A-site codon, while tRNA does not.

DNA oligonucleotides that base-pair with mRNA next to the ribosomal tunnel were tested to determine if they could: i) limit mRNA mobility; ii) make stop-codon recognition by eRF1 inefficient; and iii) make misreading the stop-codon by tRNA efficient, thus resulting in readthrough, iv) or act via a different mechanism to promote readthrough.

### III. +4-+8 Antisense Oligodeoxyribonucleotides

In one embodiment, the present invention contemplates antisense oligonucleotides (oligos) that bind to mRNA at, or between, the +4-+8 nucleotides downstream of the first nucleotide of a nonsense stop codon.

The data presented herein demonstrate that DNA antisense oligos annealing to an mRNA sequence downstream of a premature stop codon promote translation readthrough. Surprisingly, the most effective annealing site for DNA oligos start at the +8 position downstream of the premature stop codon, where the +1 position is the first nucleotide of the premature stop codon. It was observed that not all DNA oligos positioned downstream of a premature stop codon were equal in promoting readthrough. Readthrough with DNA oligos that anneal starting at positions +4 and +7 were lower than with those starting at +8, yet were more efficient than background readthrough, rendering the range of antisense oligos binding between +4 through +8 sensitive to the promotion of translation readthrough of stop codons.

Translation readthrough of a truncated protein around a premature stop codon can be different from the readthrough of a full-length protein. For example, stop codons are known to differ in their efficiency of translation termination and subsequent release of a truncated protein. For example, nonsense mutations resulting in a UGAC premature stop codon are much less efficient (e.g., a weak stop codon) in translation termination than a wild type UAAA stop codon (e.g., as strong stop codon), as a purine (A, G) nucleotide at position +4 renders translation termination more efficient than a pyrimidine (C, U) at position +4. Thus, UGAC—a “weak” stop codon—is more prone to readthrough than the “strong” UAAA stop codon. Indeed, most studies testing small molecules report most efficient readthrough of the UGAC stop codon, while UAAA or UGAG can be completely resistant to readthrough.

In one embodiment, the present invention contemplates antisense oligonucleotides that efficiently readthrough and translate a functional protein from mRNAs with “weak” premature stop codons and/or mRNAs with “strong” stop codons either individually or in combination with an aminoglycoside. The data suggest that antisense oligonucleotides complementary to a mRNA nucleotide sequence at +4-+8 positions downstream of the first nucleotide of a premature stop codon are effective therapeutic candidates for genetic diseases caused by nonsense mutations. These findings show that DNA complementarity of an aberrant mRNA is specific, yet versatile, as antisense oligos or other nucleic acid sequences (i.e., RNA, LNA and other modifications) can bind to mRNAs having premature stop codons that cause various diseases.

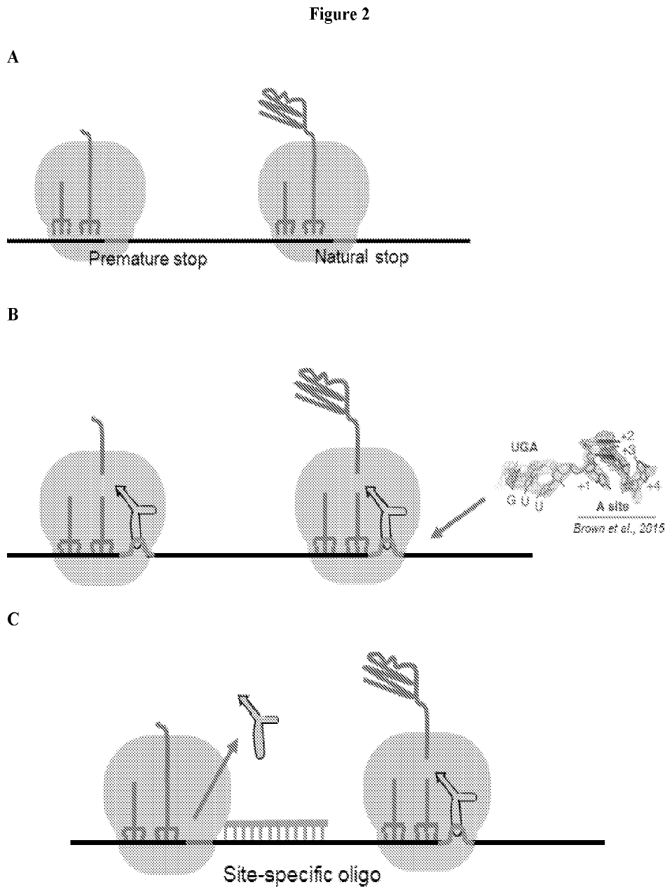

Some genetic diseases are caused by an mRNA molecule having a premature stop codon because a truncated protein is translated and released. In contrast, if an mRNA molecule has a natural (wild type) stop codon in its proper position a full-length protein is translated and released. See, FIG. 2A. Translation termination protein factors function to bind to, and “pull” stop codons into a ribosome resulting in the release of proteins from ribosomes which prevents further mRNA translation. See, FIG. 2B. Site-specific antisense oligos positioned downstream from premature stop codon prevent “pulling” of the stop codon into the ribosome and thus inhibit early protein translation termination leading to readthrough of a premature stop codon and translation of a full-length protein. See, FIG. 2C.

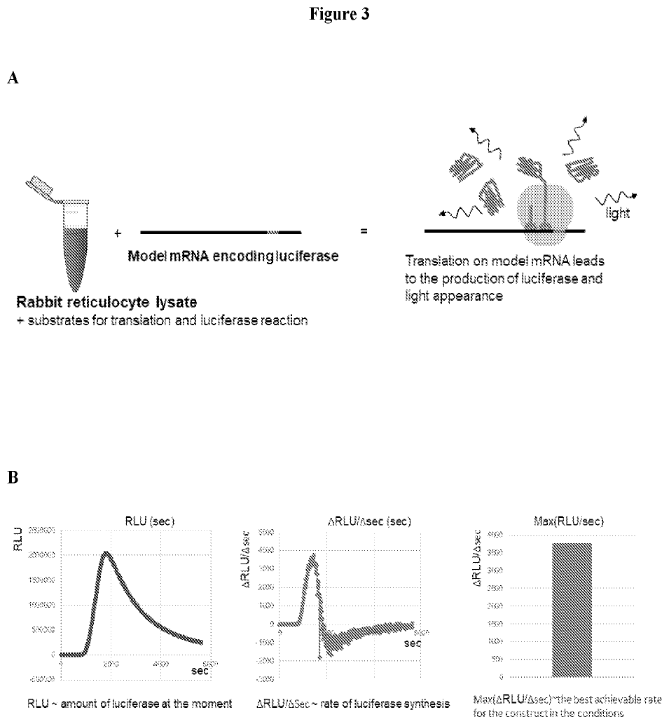

The data presented herein was collected using a translation assay with a commercial cellular extract (i.e., a rabbit reticulocyte lysate). See, FIGS. 3A and 3B. Preliminary data found that antisense oligos placed at a +8 nucleotide position from an RNA premature stop codon (with +1 position corresponding to the U of the stop codon) resulted in superior readthrough of two different mRNA sequences with premature stop codons, while other downstream position placements resulted in less efficient readthrough. Further, the data show that readthrough efficiency is substantially increased by a combination of the antisense oligo with low concentrations of an aminoglycoside (e.g., G418) thereby providing restoration of 30-40% of functional protein translation.

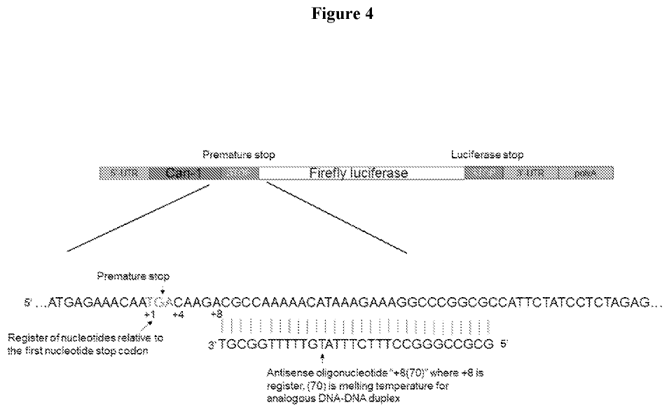

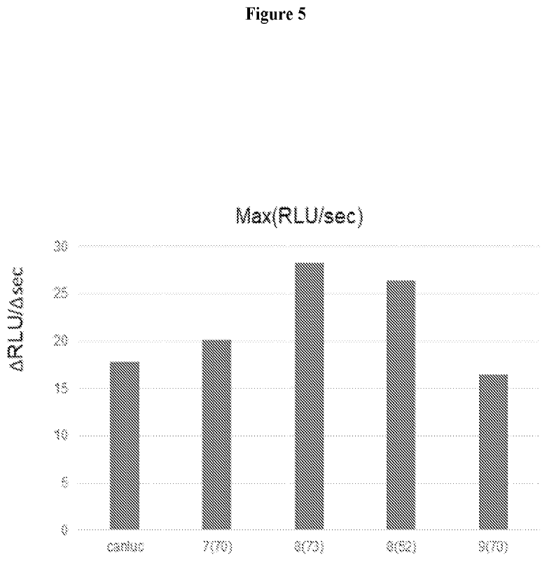

The DNA antisense oligos contemplated herein were validated using a DNA expression construct comprising a premature stop codon and a luciferase gene. The basic methodology was used a CAN1 gene expression construct encoding an arginine permease amino acid transporter protein. The CAN1 expression construct positions a TAG premature stop codon at the terminus of the CAN1 open reading frame subsequently followed by a luciferase open reading frame. See, FIG. 4. The DNA antisense oligo +8(70) having the nucleic acid sequence of 5′-GCGCCGGGCCTTTCTTTATGTTTTTGGCGT-3′ was then positioned at the +8 nucleic acid position downstream of the first nucleotide of the premature stop codon. The data show that +8 antisense oligos positioned downstream to a premature stop increases readthrough of a premature stop codon in a CAN1-1uc DNA expression construct. See, FIG. 5.

### IV. Treatment Of Cystic Fibrosis With Antisense Oligos

In one embodiment, the present invention contemplates a plurality of specific +4 through +8 (e.g., +4, +5, +6, +7, +8) antisense oligomers that are useful in the treatment of cystic fibrosis.

Cystic fibrosis is an inherited life-threatening disorder that damages the lungs and digestive system. In particular, cystic fibrosis affects the cells that produce mucus, sweat, and digestive juices and causes these fluids to become thick and sticky. The causative factor of cystic fibrosis is believed to be a genetic mutation G542X having a population frequency of ˜2.4%,

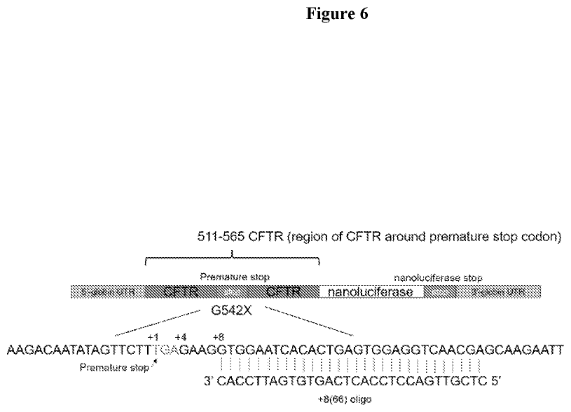

In one embodiment, the present invention contemplates a modified cystic fibrosis transmembrane conductance regulator (CFTR) deoxyribonucleic acid (DNA) construct. In one embodiment, the modified CFTR DNA construct comprises a CFTR open reading frame comprising a premature TGA stop codon. In one embodiment, the TGA premature stop codon is a G542X mutation. In one embodiment, the modified CFTR DNA construct further comprises a nanoluciferase gene and a natural stop codon. In one embodiment, the modified CFTR mRNA construct molecule is hybridized to a single stranded DNA antisense oligo at the +8 nucleotide position downstream of a TGA premature stop codon. In one embodiment, the DNA antisense oligo is 5′-CTCGTTGACCTCCACTCAGTGTGATTCCAC-3′. See, FIG. 6.

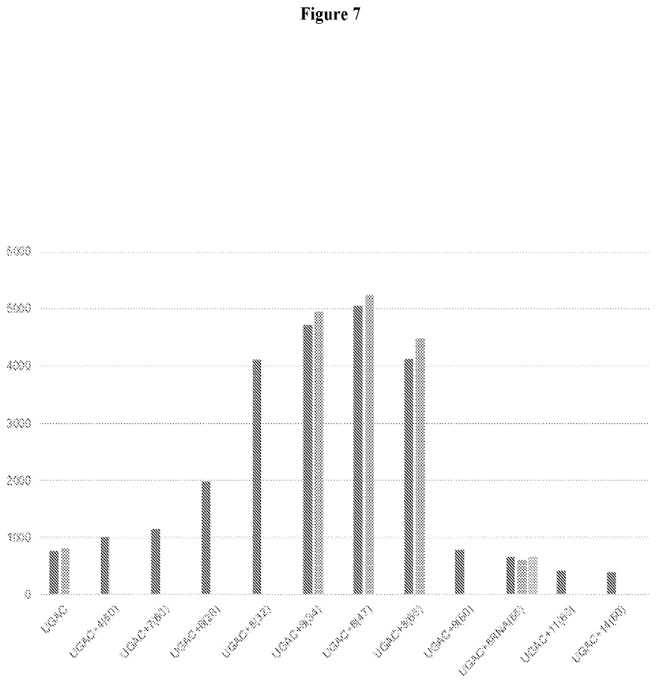

A 511-565 CFTR DNA expression vector construct was designed with several different codon configurations: i) a single wild type sense codon (“GGAG”) and a subsequent wild-type stop codon; ii) a premature stop codon “TGAG” comprising a G542X mutation (e.g., a strong stop codon); and iii) a premature stop codon “TGAC” comprising a G542X mutation (a weak stop codon) rendering an artificial readthrough-prone context. The data showed that the CFTR DNA expression vector construct with the “TGAC” premature stop codon provided a DNA antisense oligo hybridizing at the +8 nucleotide position downstream of the “TGAC” premature stop codon and showed significant translation readthrough as compared to +4, +7, +9, +11 and +14 nucleic acid downstream positions. Surprisingly, the +8(32), +8(34), +8(47) and +8(66) DNA antisense oligos showed between 4-5 fold higher readthrough as compared to the other downstream positions. Nevertheless, antisense oligo hybridized to positions +4 and +7 showed progressively higher readthrough than background readthrough, whereas oligos hybridizing at positions +9, +11 and +14 showed reduced readthrough. See, FIG. 7.

The CFTR premature stop codon DNA construct expression readthrough analysis was also performed with the aminoglycoside G418, having the structure of:

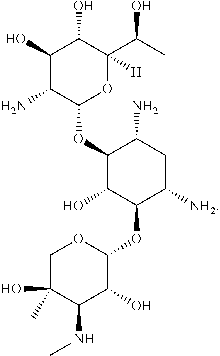 [SMILES: CN[C@@H]1[C@@H](O)[C@@H](O[C@H]2[C@H](O)[C@@H](O[C@H]3O[C@H]([C@H](C)O)[C@@H](O)[C@H](O)[C@H]3N)[C@H](N)C[C@@H]2N)OC[C@]1(C)O]

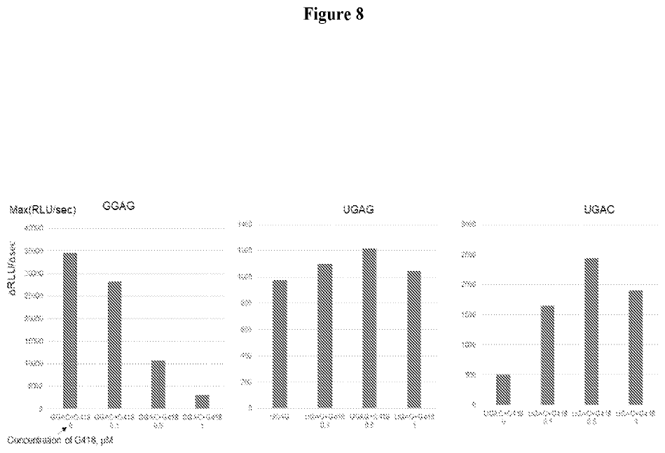

The data showed that G418 inhibits wild type sense codon (GGAG) readthrough in a concentration-dependent manner. Additionally, G418 promotes premature stop codon (UGAG and UGAC) readthrough in a concentration-dependent manner. See, FIG. 8.

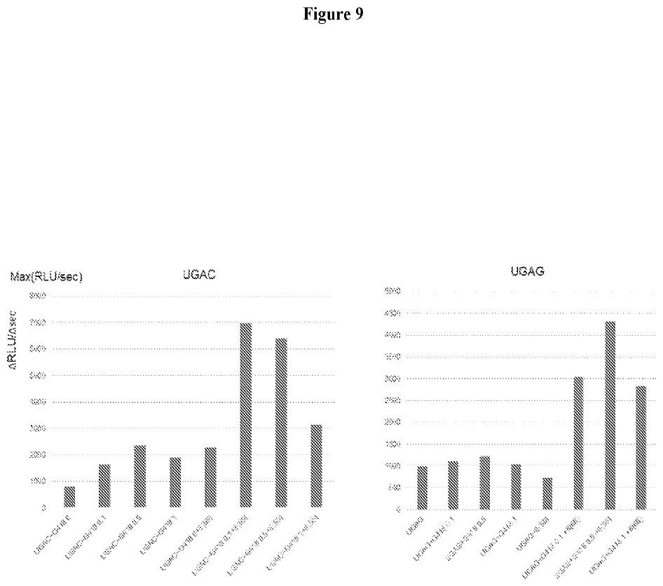

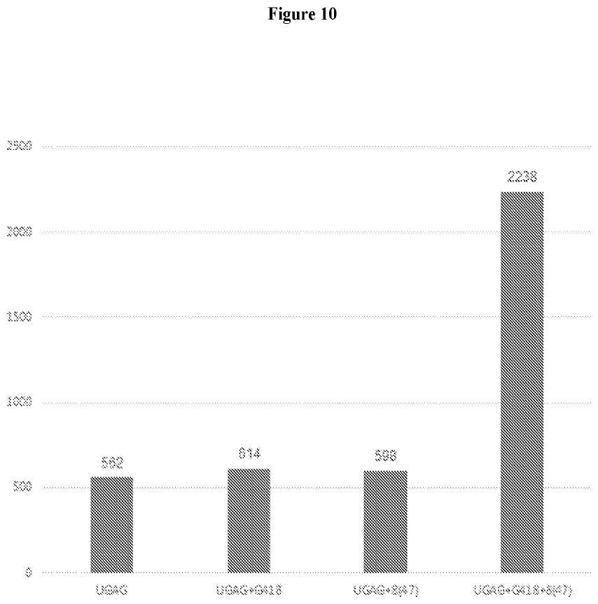

Because aminoglycosides are used in the clinical therapy of some premature stop codon-associated diseases, DNA antisense oligos and G418 were evaluated for a synergistic effect. The data show that a combination of a +8(66) antisense oligo and G418 do provide a synergistic effect of up to 5-fold promoting readthrough of premature stop codon using a CFTR DNA expression construct, when compared to either G418 or a +8(66) DNA oligo alone. See, FIG. 9. A strong UGAG (patient mutation context) premature stop codon is harder to readthrough than a UGAC premature stop codon. Surprisingly, the 0.5 μM and 0.1 μM G418 concentrations had a significantly higher readthrough rate than the 1 μM G418 concentration of both the weak (UGAC) and strong UGAG premature codon (patient mutation context). Similar synergistic data was observed when using G418 and a +8(47) DNA antisense oligo in combination as opposed to alone with the CFTR UGAG mRNA. See, FIG. 10.

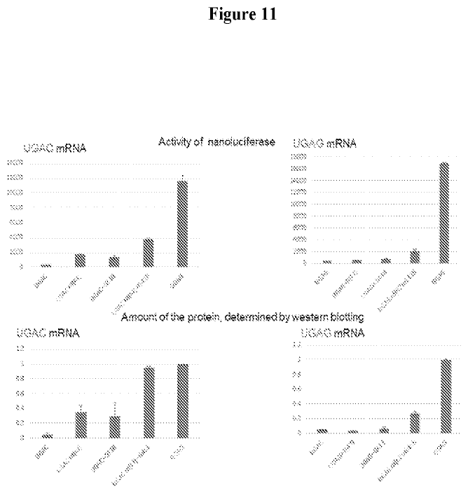

This mRNA readthrough induced by the presently disclosed antisense oligos was validated by correlating nanoluciferase activity with expressed protein level. See, FIG. 11. These data show that translation readthrough as revealed by increased luciferase signal correlates with a proportional increased amount of full-length protein.

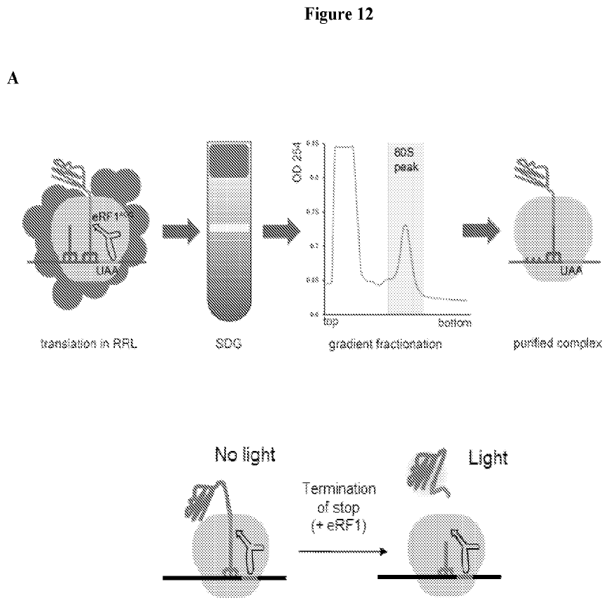

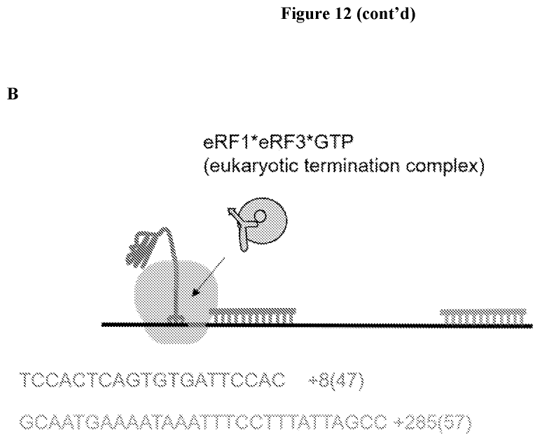

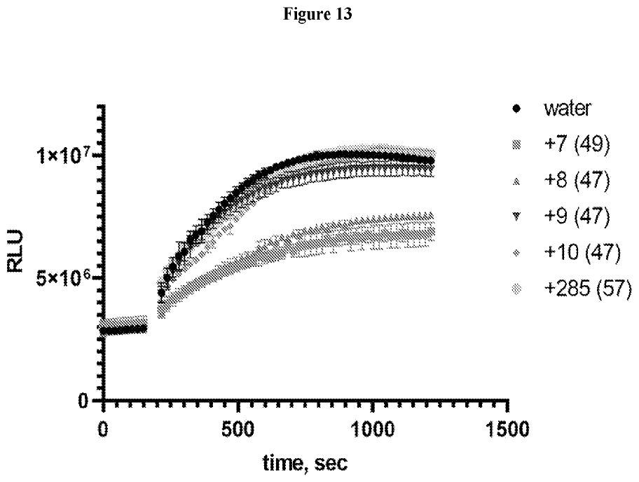

The termination step during an induced oligo translation readthrough of a stop codon was assessed with a modified luciferase assay (e.g., Termiluc). In brief, this assay identifies translation termination upon the appearance of light in the presence of an eRF1 eukaryotic termination protein. See, FIGS. 12A and 12B. The data show that readthrough promoter oligonucleotides do inhibit termination translation in vitro in sequence specific-manner. See, FIG. 13.

Termination of translation was accompanies by an inhibited protein release as shown with antisense oligos +7 and +8 using a Termiluc assay. See, FIG. 13. These data are consistent with readthrough data obtained in a rabbit reticulocyte lysate (RRL) assay. Additionally, the +9 antisense oligo was least efficient, also consistent with the RRL data.

These data suggest that the antisense oligos as contemplated herein specifically interfere with the translation termination step, thereby lowering the release of truncated protein and allowing the ribosome to continue translation. Although it is not necessary to understand the mechanism of an invention, it is believed that the presently disclosed stop codon readthrough technology is programmable to provide specificity for a particular stop codon of a particular mRNA.

### V. Modified Antisense Oligos

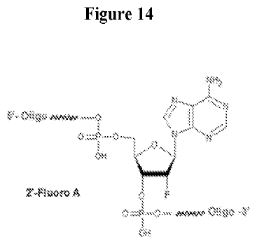

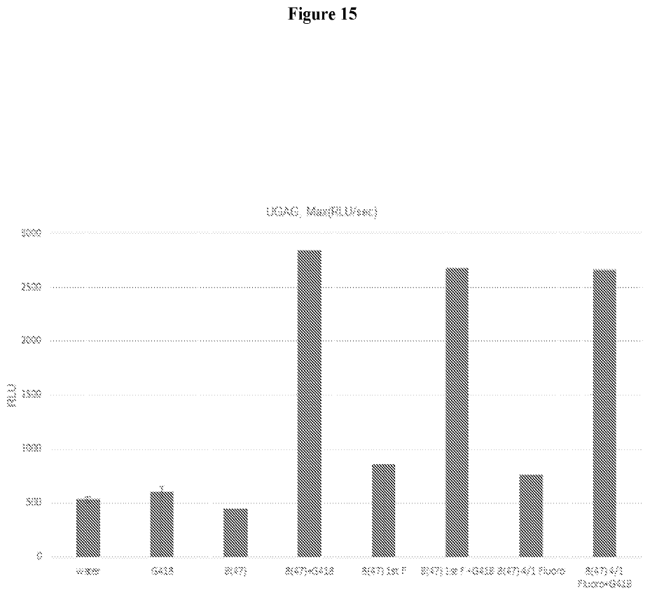

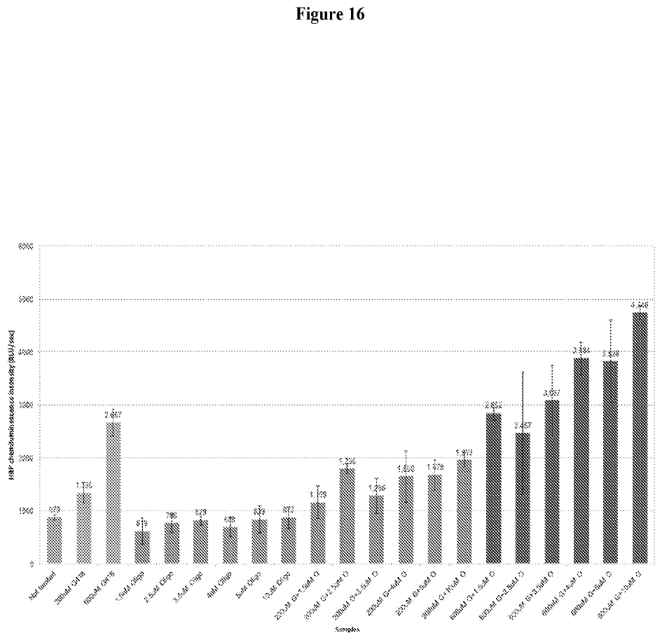

In one embodiment, the present invention contemplates a +4-+8 antisense oligomer readthrough promoter comprising at least one ribose modification. In one embodiment, these modification include, but are not limited to, a 2′-fluoride (F) modification, a 2′-O-methyl (Ome) modification and a phosphothioate (PS) linkage modification. See, FIG. 14. For example, +8(47) oligos were modified with a fluoride at least one nucleic acid (NA^{F}): i) one modification: TCCACTCAGTGTGATTCCAC^{F}; and ii) six modifications: U^{F}CCAC^{F}TCAG^{F}TGTG^{F}ATTC^{F}CAC^{F}. The data presented herein demonstrates that these modified antisense oligos promote readthrough in an RRL assay with improved stability and/or efficiency, and in mammalian cell culture. See, FIG. 15 and FIG. 16.

Fischer rat thyroid (FRT) cells expressing CFTR (G542X) and horseradish peroxidase (HRP) fusion proteins were cultured at 37° C. and 5% CO₂ in a Ham's F-12, Coon's Modification (Sigma-Aldrich, St. Louis, MO, #F6636) buffer with 10% fetal bovine serum (FBS; Thermo Fisher Scientific, #26140-079), 1% penicillin-streptomycin (Thermo Fisher Scientific, #15140-122), and 100 μg/mL hygromycin B (Thermo Fisher Scientific, #10687010). Then, these FRT cells were seeded at a density of 2×10⁴ cells/well in Costar 96 well plate.

Twenty-four (24) hours after seeding, FRT cells expressing CFTR (G542X) were incubated for another twenty-four (24) hours with or without different concentrations of G418 and/or the following modified 8(47) antisense oligo:

U^{F}CCAC^{F}TCAG^{F}TGTG^{F}ATTC^{F}CAC^{F}

An HRP luminescence assay was then performed after the cells were washed three times with 1×DPBS, followed by incubation with SuperSignal West Femto HRP substrate (15 μL/well, Thermo Fisher Scientific, #34096) for 5 min at room temperature. The HRP-catalyzed luminescence was read with a Tecan microplate reader under the following conditions: room temperature; no shake/no delay; integration time, 0.1 s; read height, 8.00 mm. The data show that a combination of G418 and the oligo results in a dose-response related synergy of nonsense codon readthrough. See, FIG. 16.

These data suggest that a chemically-modified oligo, together with G418, improves the readthrough of premature stop codon in cells expressing a disease-causing variant of CFTR (with G542X mutation).

### VI. Treatment Of Rett Syndrome With Antisense Oligos

In one embodiment, the present invention contemplates a plurality of specific +4 through +8 (e.g., +4, +5, +6, +7, +8) antisense oligomers that are useful in the treatment of Rett syndrome.

Rett syndrome is a rare genetic mutation affecting brain development that has primarily been found in females. Briefly, infants seem healthy during their first six months, but over time, rapidly lose coordination, speech, and use of the hands. Symptoms may then stabilize for years. There's no cure, but medications, physical and speech therapy, and nutritional support help manage symptoms, prevent complications, and improve quality of life. Recently, a genetic basis has been found that appears to involve premature stop codons.

It is believed that a dysfunctional brain protein, methylcytosine-binding protein 2 (Mecp2), may be responsible for many of the Rett syndrome symptoms. The MECP2 gene provides instructions for making a protein called MeCP2. This protein helps regulate gene activity (expression) by modifying chromatin, the complex of DNA and protein that packages DNA into chromosomes. The MeCP2 protein is present in cells throughout the body, although it is particularly abundant in brain cells.

In the brain, the MeCP2 protein is important for the function of several types of cells, including nerve cells (neurons). The protein likely plays a role in maintaining connections (synapses) between neurons, where cell-to-cell communication occurs. Many of the genes that are known to be regulated by the MeCP2 protein play a role in normal brain function, particularly the maintenance of synapses.

Those skilled in the art believe that the MeCP2 protein may also be involved in processing molecules called messenger RNA (mRNA), which serve as genetic blueprints for making proteins. By cutting and rearranging mRNA molecules in different ways, the MeCP2 protein controls the production of different versions of certain proteins. This process is known as alternative splicing. In the brain, the alternative splicing of proteins plays a role in normal communication between neurons and may also be necessary for the function of other types of brain cells.

Most cases of Rett syndrome are caused by a mutation in methylcytosine-binding protein 2 (MECP2) gene. The MECP2 gene is located on the X chromosome. Between 90% and 95% of girls with Rett syndrome have a mutation in the MECP2 gene. Amir et al., “Rett syndrome is caused by mutations in X-linked MECP2” Nature Genetics 23(2): 185-188 (1999); Schollen et al., “Gross rearrangements in the MECP2 gene in three patients with Rett syndrome: Implications for routine diagnosis of Rett syndrome” Human Mutations 22:116-120 (2003); and Zoghbi, H. Y. “MeCP2 dysfunction in humans and mice” Journal of Child Neurology 20:736-740 (2005). It is generally believed that about eight (8) mutations in the MECP2 gene are responsible for the most prevalent causes of Rett syndrome. The development and severity of Rett syndrome symptoms depend on the location and type of the mutation on the MECP2 gene. Percy et al., “Rett syndrome: North American database” Journal of Child Neurology 22(12): 1338-1341 (2007).

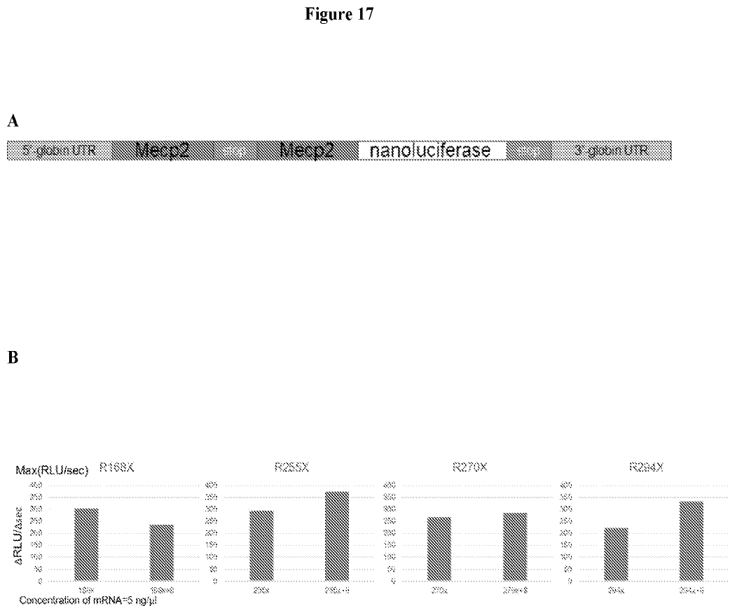

The data presented herein use an Mecp2 DNA expression vector construct that expresses an Mecp2 mRNA with one of four (4) premature stop codons comprising nonsense mutations (e.g., R168X, R255X, R270X or R294X). See, FIG. 17A. The data show that a +8 DNA antisense oligo, only mildly enhanced readthrough of mRNAs at three out of four premature stop codons. See, FIG. 17B.

### EXPERIMENTAL

### Example 1

### Cell Culture

A. Exemplary Cell Lines
CFF-16HBEge CFTR G542X
CFF-16HBEge CFTR R1162X
CFF-16HBEge CFTR W1282X
CFFT-16HBEge CFTR Y122X

B. Exemplary Cell Culture Conditions
Medium (Store complete medium at 4° C.)

| Component | CFF Vendor/Catalog | % (final) |
|---|---|---|
| Minimum Essential | Gibco 11095-072 | 89% |
| Medium | | |
| Fetal bovine serum | Gibco 26140-079 | 10% |
| Penicillin/Streptomyc | Gibco 15140-122 | 1% |
| in (100x) | | |

Freezing Solution (Make fresh)

| Component | % (final) |
|---|---|
| Complete media | 40% |
| (see above) | |
| Fetal bovine serum | 50% |
| DMSO | 10% |

C. Other Reagents
DPBS (Hyclone SH30028.02)
TrypLE Express (Gibco 12604-021)

D. Coating Solution (Make fresh)

| | | | |
|---|---|---|---|
| Component | CFF Vendor/Catalog | 50 | mL final solution |
| LHC-8 basal medium | Gibco 12677-027 | 48 | mL |
| Bovine serum albumin | Gibco 15260-037 | 67 | μL |
| 7.5% | | | |
| Bovine collagen solution, | Advanced BioMatrix | 0.5 | mL |
| Type 1 | 5005-100 ML | | |
| Fibronectin from human | Thermo Fisher Scientific | 0.5 | mL |
| plasma, 1 mg/ml | 33016-015 | | |

Coat flasks with the coating solution: 1 ml of the solution for a T-25 flask, 2 ml for a T-75.
Distribute the solution evenly across the surface, making sure the entire surface is wetted by the
solution and leave for 2-3 hours at 37° C. After incubation, thoroughly remove liquid. Do not
reuse this solution. Do not rinse the containers. The coated flasks can be stored at 4° C. for several
months.

E. Exemplary Sub-Culturing Protocol
Sub-culture at 80-100% confluency
Remove medium and rinse with DPBS and remove
Add TrypLE Express (spread across the cell surface) and incubate the flask at 37° C.
After 3-5 minutes, examine the flask under a microscope to observe the degree of cell
detachment. If the cells are coming off the surface use the palm of your hand and hit
against the side of the flask to facilitate further detachment. If the cells are still attached,
put the flask back into the incubator for a few more minutes and examine the flask again.
The goal is to remove >95% of the cells from the surface.
Pipette the cells repeatedly to break up any clumps and transfer cell suspension into a
centrifuge tube.
Spin for 3.5 minutes at 1200 rpm to pellet the cells Note: If you cannot spin the cells
(such as high throughput 96 well format), this step can be skipped
Discard the liquid and resuspend the cell pellet in complete medium.
Split cells at 1:10 to 1:20 for one week passages CFF media change schedule - 3×
weekly
F. Exemplary Liquid Nitrogen Storage Protocol
Treat the cells as for sub-culturing except resuspend the cell pellet in freezing solution.
Transfer the cells into freezing vials, 1-1.5 mL/vial. For a T-75 flask, aliquot into 4-6
vials, a T-75 into 2-4 vials depending on cell density. CFF freezes 5 million cells/vials
for thawing into a T-75 flask.
Put the vials in controlled cooling chamber and plate in −80° C. freezing overnight
(the chamber regulates cooling at ~1° C./minute)
Move the vials from the cooling chamber into liquid nitrogen storage as quickly
as possible
G. Exemplary Culturing Frozen-Stored Cells
Remove a vial of cells from liquid nitrogen and thaw the cells in a 37° C. water bath
Resuspend with 5 mL complete medium
Spin for 3.5 minutes at 1200 rpm to pellet the cells
Remove liquid (to remove DMSO)
Resuspend cell pellet with 9 mL medium (for T-75 flask) and seed the cells onto a
coated flask
Grow cells in 37° C. incubator with 5% CO2.
After overnight incubation, the cells should attach to the flask. The rounded, un-
attached cells are dead cells.

### Example II

### Gene Editing

A.
Cas9 Protein Complex
An RNP complex including:
Cas9: S. pyogenes Cas9
Guide RNA target sequence:
5′-GAGAAAGACAATATAGTTCT-3′
B.
Protospacer accessory motif sequence:
TGG
C.
Homology Directed Repair donor
template sequence (ssODN: 5′-3′):
GCAAATGCTTGCTAGACCAATAATTAGTTATTC
ACCTTGCTAAAGAAATTCTTGCTCGTTGACCTC
CACTCAGTGTGATTCCACCTTCTCAAAGAACTA
TATTGTCTTTCTCTGCAAACTTGGAGAT
D.
Amplifying/Sequencing Primers
Forward Primer:
5′-ATGGAAGCCCAGTGAAGATAC-3′
Reverse Primer:
5′-CTAGCCATAAAACCCCAGGA-3′

### Example III

### Sequencing

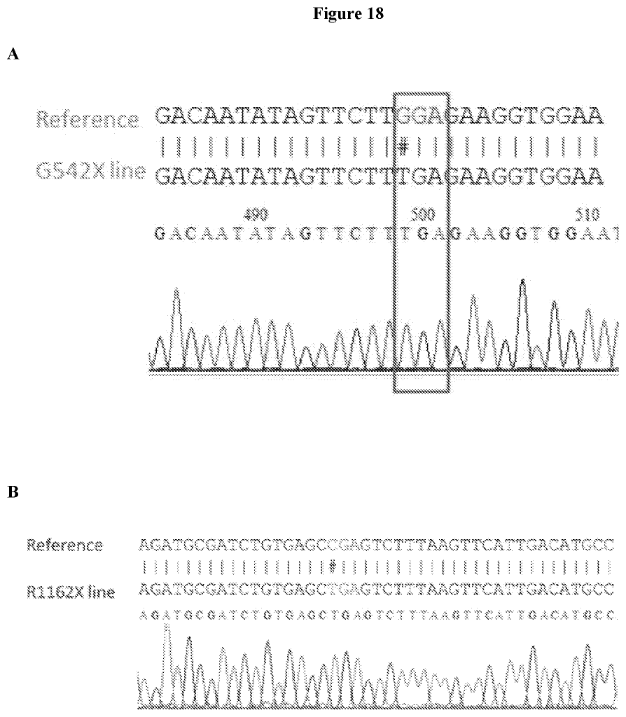

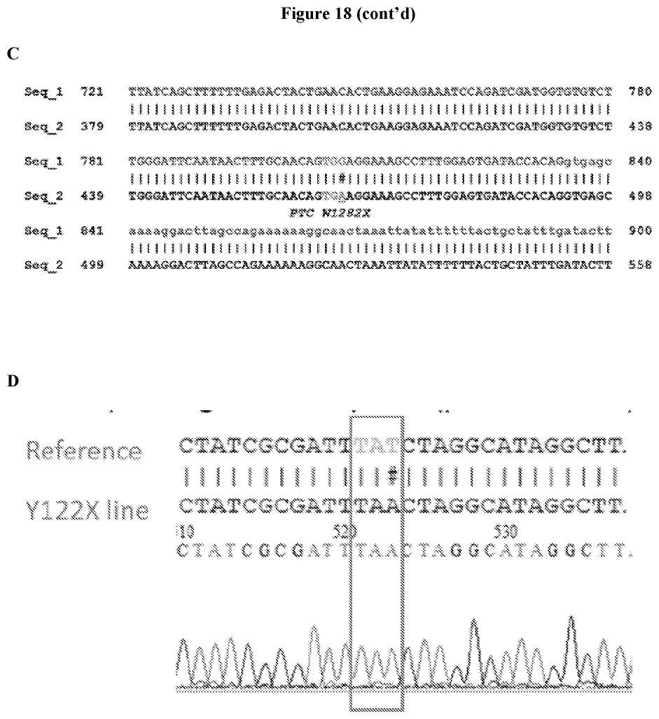

Bulk sequencing of PCR amplicons was performed using the primers in accordance with Example II. A TOPO cloning (e.g, allelic exclusion) method sequenced the plasmid vector. See, FIG. 18A-D.

The data shows that no large deletions or re-arrangements were identified with next generation sequencing of the CFTR locus. However, an insertion of unknown size was observed at position (hg38) Chr7:117536118 in intron 6 of one CFTR allele in the 16HBE14o-parental cells and the 16HBEge cells that were derived from them. This insertion contains SV40 genomic sequence, which was used in the immortalization process to create the 16HBE14o-cells and is not a result of gene editing. Several lines of evidence support that the allele carrying the insertion yields a degraded CFTR transcript or non-functional CFTR; therefore, the 16HBE14o-cells are functionally mono-allelic. The 16HBEge cell lines are homozygous for the engineered CFTR variant and express CFTR from the same number of alleles as the 16HBE14o-cells.

### Example IV

### Gene Expression

The example presents the results of mRNA analysis by quantitative polymerase chain reaction and (qPCR) and protein analysis by Western Blot.

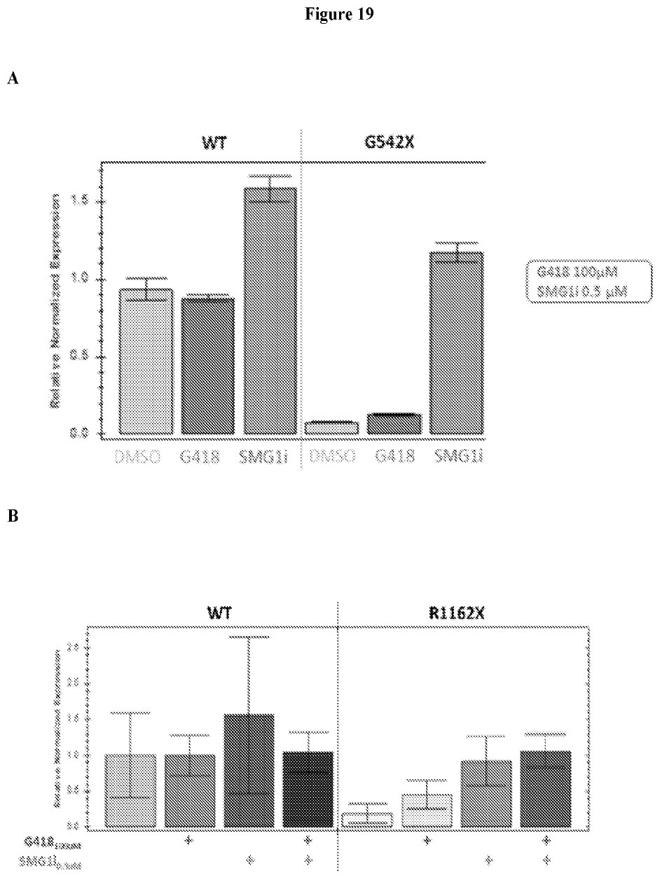

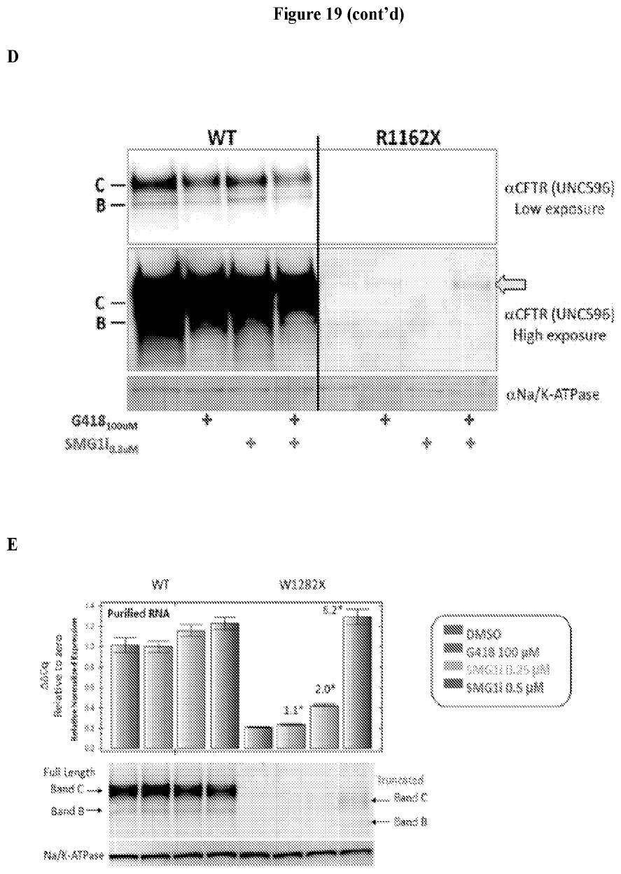

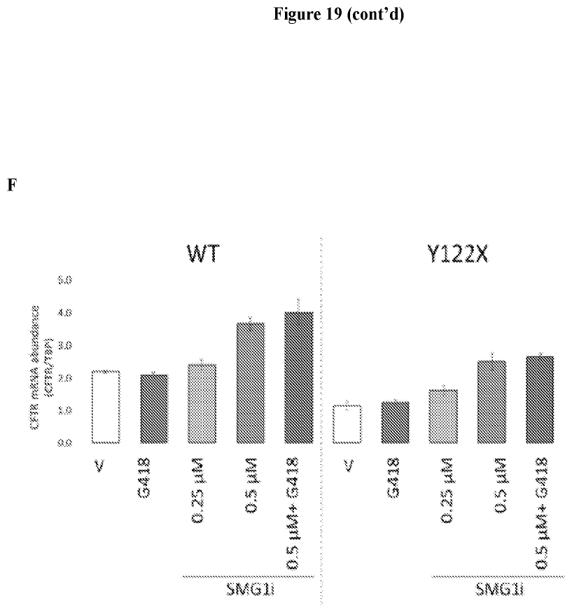

The data shows that the antisense, SMG1i, is a pharmacological inhibitor of the NMD-mediator SMG1 and restores mRNA levels of the PTC alleles. As usual, a smaller increase in CFTR WT mRNA is also observed with SMG1i. See, FIG. 19A-F.

### Example V

### Electrophysiology

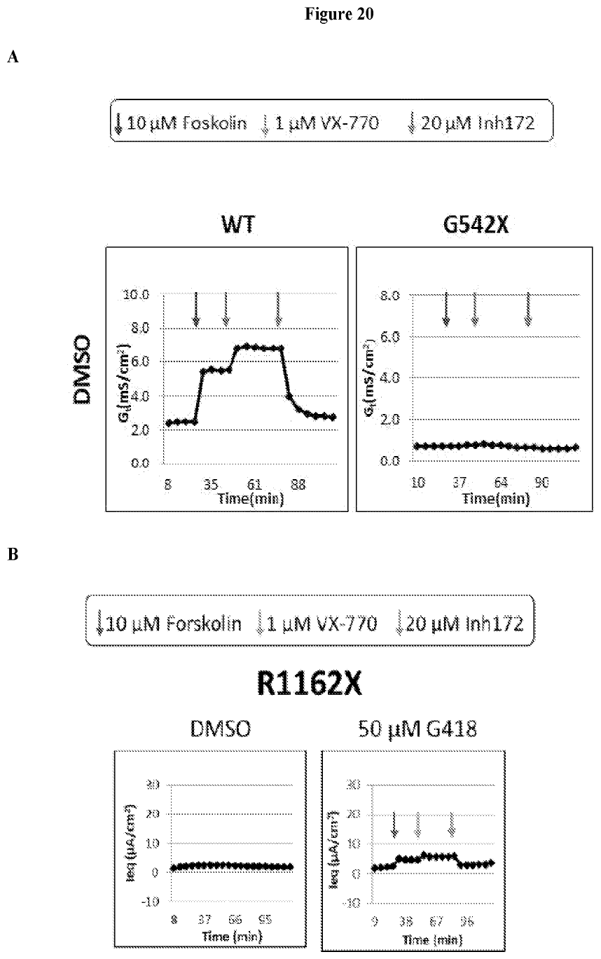

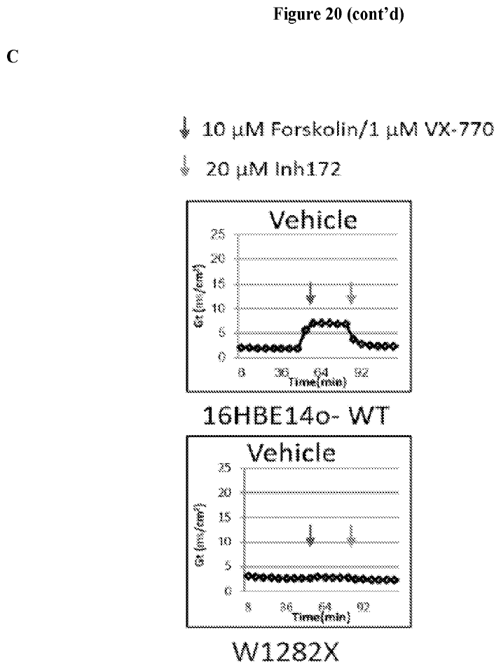

A functional assay was performed at one (1) week post-filter seeding using a TECC/24 Conductance Assay that demonstrated resistance but no measurable CFTR function. See, FIGS. 20A-C.

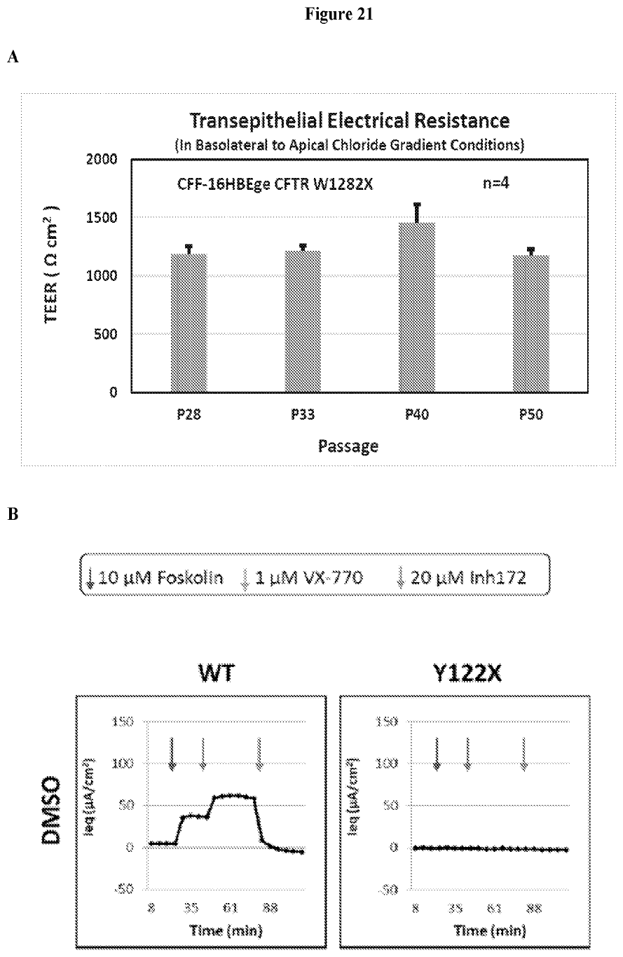

The transepithelial resistance of the CFF-16HBEge lines is stable over time (up to passage 50 tested in the CFF-16HBEge CFTR W1282X cell line). See, FIGS. 21A-B.

1-21. (canceled)

22. A composition comprising: i) a deoxyribonucleic (DNA) or modified nucleic acid antisense oligomer; and ii) a messenger ribonucleic acid (mRNA) molecule encoding an RNA sequence and a premature stop codon, wherein said antisense oligomer is complementary to said RNA sequence starting between a +4-+8 nucleotide position downstream of the first nucleotide of said premature stop codon.

23. The composition of claim 22, wherein said antisense oligomer is hybridized to said RNA sequence.

24. The composition of claim 22, wherein said mRNA molecule encodes a cystic fibrosis transmembrane conductance regulator protein (CFTR) or a methylcytosine-binding protein 2 protein.

25. (canceled)

26. The composition of claim 22, wherein said composition further comprises an aminoglycoside.

27-28. (canceled)

29. The composition of claim 22, wherein said aminoglycoside is selected from the group consisting of G418, gentamicin, amikacin, tobramycin, kanamycin, streptomycin and neomycin.

30. The composition of claim 22, wherein said premature stop codon is selected from the group consisting of UGAC, UGAG, UGAA and UGAT.

31-34. (canceled)

35. The composition of claim 22, wherein said oligomer is a nucleic acid having the sequence selected from the group consisting of
CCTCCACTCAGTGTGATTCCACCTTC,
CCACTCAGTGTGATTCCACC,
GACCTCCACTCAGTGTGATTCCACC,
CTCAGTGTGATTCCAC,
ACTCAGTGTGATTCCAC,
TCCACTCAGTGTGATTCCAC,
CTCGTTGACCTCCACTCAGTGTGATTCCAC,
CTGAAGCTGACCCTCAGGCC,
CGGGGAGTGTGGTGGCAG
and
TGCAGGAGACCGTACTCCCC.

36-44. (canceled)

45. The composition of claim 22, wherein said oligomer comprises at least one nucleotide with a modification.

46. The composition of claim 45, wherein said modification is selected from the group consisting of a 2′-fluoride (F) modification, a 2′-O-methyl (Ome) modification and a phosphothioate (PS) linkage modification.

47-49. (canceled)

50. A method, comprising:
a) providing;
i) a patient comprising a messenger ribonucleic acid (mRNA) molecule with a premature stop codon and exhibiting at least one symptom of medical disorder; and
ii) a pharmaceutically acceptable composition comprising a deoxyribonucleic (DNA) or modified nucleic acid antisense oligomer that is complementary to a mRNA sequence starting between a +4-+8 nucleotide position downstream of the first nucleotide of said premature stop codon; and
b) administering said pharmaceutically acceptable composition to said patient such that said at least one symptom of said medical disorder is reduced.

51. The method of claim 50, wherein said medical disorder is caused by said premature stop codon.

52. The method of claim 50, wherein said medical disorder is cystic fibrosis or Rett syndrome.

53. The method of claim 50, wherein said pharmaceutically acceptable composition further comprises an aminoglycoside.

54. The method of claim 53, wherein said administering does not result in aminoglycoside side effects.

55. (canceled)

56. The method of claim 53, wherein said aminoglycoside is selected from the group consisting of G418, gentamicin, amikacin, tobramycin, kanamycin, streptomycin and neomycin.

57. The method of claim 50, wherein said mRNA molecule encodes a cystic fibrosis transmembrane conductance regulator protein or a methylcytosine-binding protein 2 protein.

58. (canceled)

59. The method of claim 50, wherein said premature stop codon is selected from the group consisting of UGAC, UGAG, UGAA and UGAT.

60-63. (canceled)

64. The method of claim 50, wherein said oligomer is a nucleic acid having the sequence selected from the group consisting of
CCTCCACTCAGTGTGATTCCACCTTC,
CCACTCAGTGTGATTCCACC,
GACCTCCACTCAGTGTGATTCCACC,
CTCAGTGTGATTCCAC,
ACTCAGTGTGATTCCAC,
TCCACTCAGTGTGATTCCAC,
CTCGTTGACCTCCACTCAGTGTGATTCCAC,
CTGAAGCTGACCCTCAGGCC,
CGGGGAGTGTGGTGGCAG
and
TGCAGGAGACCGTACTCCCC.

65-73. (canceled)

74. The method of claim 50, wherein said oligomer comprises at least one nucleotide with a modification.

75. The method of claim 74, wherein said modification is selected from the group consisting of a 2′-fluoride (F) modification, a 2′-O-methyl (Ome) modification and a phosphothioate (PS) linkage modification.

76-109. (canceled)
